(19) United States
(12) Patent Application Publication
(10) Pub. No.: US 2004/0142495 A1
(43) Pub. Date: Jul. 22, 2004

(54) Analyte detecting article and method

(76) Inventors: William G. Hartman, North Royalton, OH (US); Nina Patel-Lahanis, Medina, OH (US); Kai Li, Diamond Bar, CA (US); Daniel L. Holguin, Fullerton, CA (US); Richard L. Sandt, Brunswick, OH (US); Charles K. Herrmann, Cleveland Heights, OH (US)

Correspondence Address: Heidi A. Boehlefeld, Renner, Otto, Boisselle & Sklar, LLP, Nineteenth Floor, 1621 Euclid Avenue, Cleveland, OH, 44115-2191, US

(21) Appl. No.: 10/737,023

(22) Filed: Dec. 16, 2003

Related U.S. Application Data
(60) Provisional application No. 60/433,737, filed on Dec. 16, 2002.

(51) Int. Cl. G01N 33/543

(52) U.S. Cl. 436/518

(57) ABSTRACT

An article for detecting the presence, or the absence, of an analyte. The analyte is indicative of a change in the status of a packaged material. The article generally includes a facestock film having first and second surfaces, an adhesive layer adjacent to the facestock film, and a detecting system adjacent to the facestock film. A measurable analyte can be in vapor and/or liquid form, and the detecting system indicates whether the analyte is present. That is, the detecting system responds to contact with the analyte by indicating that such contact has occurred, or that the analyte is present.

### CROSS-REFERENCE TO RELATED APPLICATIONS

This application claims priority to provisional application 60/433,737 filed Dec. 16, 2002, the content of which is hereby incorporated by reference in its entirety.

### BACKGROUND OF THE INVENTION

1. Field of Invention

The present invention relates generally to an article that can detect an analyte, the presence of which indicates a condition of packaged material. More particularly, the invention is directed to a label for incorporation into a food package that indicates the presence of undesirable bacteria, food borne pathogens and/or spoilage.

2. Discussion of Related Art

Food related illnesses are generally considered undesirable, particularly for children and the elderly, who can have susceptible immune systems. The food services industry has developed devices and articles for determining whether food is fresh and/or is safe for consumption, and implemented processes and practices to reduce or eliminate food related illnesses.

Biological organisms responsible for most food decay often cause foul odors or off coloring. Accordingly, some consumers test food for freshness or safety by visual inspection or by smelling the food. Unfortunately, at least some pathogenic bacteria are generally undetectable by visually inspection or by smell. Consumers who purchase perishable items that are stored at home, or are prepared at home and then stored as leftovers have very limited means to either properly package, re-package, store, or verify that the food is safe for consumption and not spoiled, or still fresh.

Relative to consumers, professional food packagers and preparers generally have more sophisticated devices and methods for determining the condition of their products and of protecting from spoilage and preserving freshness. Some conventional devices or articles currently available include time/temperature gauges or indicators, chemical sensors, gas chromatography instruments, and the like. Food manufacturers have packaging tools, materials and knowledge, which enables them to more properly package food prior to its delivery to the consumer. Proper packaging can reduce the likelihood of spoilage or extend shelf life and freshness. But, even proper packaging only delays inevitable spoilage.

Often, suppliers rely on expiry dates, or ‘best used before’ dates applied to the materials. Because the time to spoliation is variable, and is influenced by a multitude of factors, the dates can be too soon or too late relative to the actual spoiling. Thus, still fresh materials are discarded, and spoiled materials can be indicated as still being acceptable. Both situations are undesirable.

It would be desirable to have an article that provides an easy, reliable, or cost-effective way to detect if food or other perishable items, such as medicine, are fresh and useful for consumption.

### SUMMARY OF THE INVENTION

The present invention provides an article for detecting the presence, or the absence, of an analyte. The presence or absence of the analyte is indicative of a change in the status of a packaged material. For example, spoiled meat creates a measurable analyte in vapor form, thus a corresponding detector can indicate that there is no spoil indicating analyte, until such an analyte is generated by the spoilage of the packaged meat. The article includes a facestock film having first and second surfaces, an adhesive layer adjacent to the facestock film second surface, and a detecting system adjacent to the facestock film first surface. The detecting system responds to contact with the analyte by indicating that such contact has occurred, or that the analyte is present.

A configuration is provided in which a label is adhered to an inner surface of a packaging material, generally adjacent to a material, such as meat, that is being monitored for the presence of the analyte. In this embodiment, a selectively permeable membrane can allow vapor but not liquid, liquid but not vapor, or both liquid and vapor to contact the detecting system. The presence or absence of the analyte is monitored in the liquid and/or vapor and then, if the analyte is present and detected, the detecting system indicates such by, for example, changing color or becoming UV fluorescent.

In another embodiment according to the present invention, a detector system for detecting an analyte is provided. The detector system includes a polyazamacrocyclic transition metal complex; a hydrophilic and generally water insoluble binder; and a plurality of particles, preferably nano-scale alumina particles. The polyazamacrocyclic transition metal complex, the binder and the particles form a layer that is responsive to contact with the analyte by visually indicating that such contact has occurred.

### BRIEF DESCRIPTION OF THE DRAWINGS

FIG. 1A is a schematic diagram showing a side view of an embodiment in accordance with the present invention having a label incorporating an analyte detecting device.

FIG. 1B shows the label of FIG. 1A, but in which the analyte detecting device incorporates an immunoassay device.

FIG. 1C shows the label of FIG. 1B, but in which the immunoassay device has a detector antibody layer applied in register with an underlying immobilized antibody layer.

FIG. 1D shows the label of FIG. 1B, but in which the immunoassay device has a detector antibody layer applied in a pattern over the underlying immobilized antibody layer.

FIG. 2A is a schematic cross-sectional side view of an embodiment according to the present invention in which a film has surface defining micro-structures.

FIGS. 2B-2D shows alternative embodiments of FIG. 2A, in which absorbent layers are used with antibody layers.

FIG. 2E shows an alternative embodiment of FIG. 2A, having a pigmented and permeable overlayer.

FIG. 3 is an exploded cross-sectional side view of an article comprising yet another embodiment of the present invention.

FIG. 4 is a cross-sectional side view of a packet or an envelope-style detection system comprising yet another embodiment of the present invention

FIGS. 5-7 are schematic side views of articles comprising alternative embodiments of the present invention.

### DESCRIPTION OF PREFERRED EMBODIMENTS

The present invention provides generally a system for the detection of an analyte. The system includes an article that indicates the occurrence of a generally undesirable if somewhat expected change in an associated packaged material by monitoring for and detecting the presence or absence of an analyte.

In particular, the present invention provides a label that monitors the state of packaged material in a container, such as a perishable food wrapped in a plastic film, and indicates when a change, such as spoilage, occurs. As a specific example, one embodiment of the present invention provides an adhesive label that can be adhered to an inside surface of a clear, vapor-impermeable, plastic-film meat container. The label is exposed to liquid or vapor emanating from the meat in the container. Over time, the meat will become less fresh, and eventually spoil. The contents of the liquid or vapor correspond to the freshness of the meat. The label indicates the freshness of the meat by monitoring the liquid or vapor, and can expressly indicate the freshness of, or the presence/absence of spoilage in, the meat. Alternative embodiments in accordance with the present invention provide, among other things, differing methods of detection, differing transmission methods of an analyte to a monitoring and detecting device, differing methods of indication, and differing configurations and placements of the detection system. Accordingly, the following illustrated embodiments define various features in accordance with the present invention.

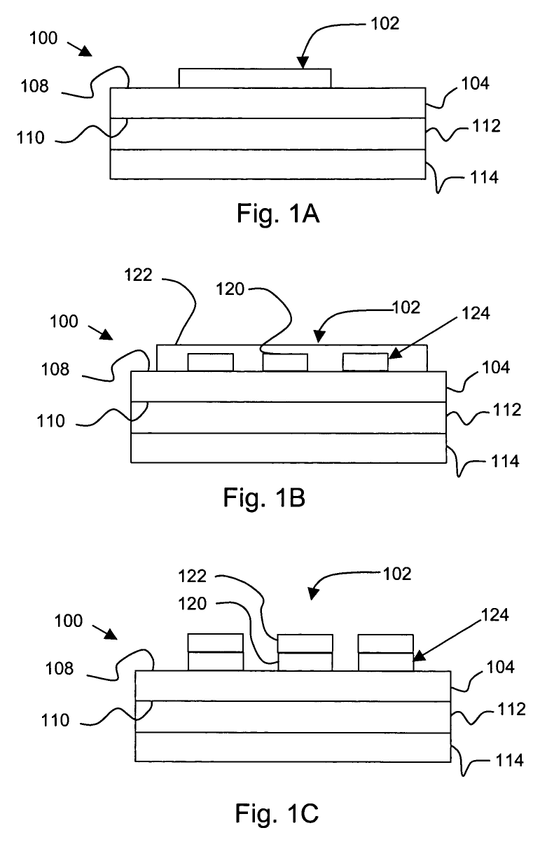

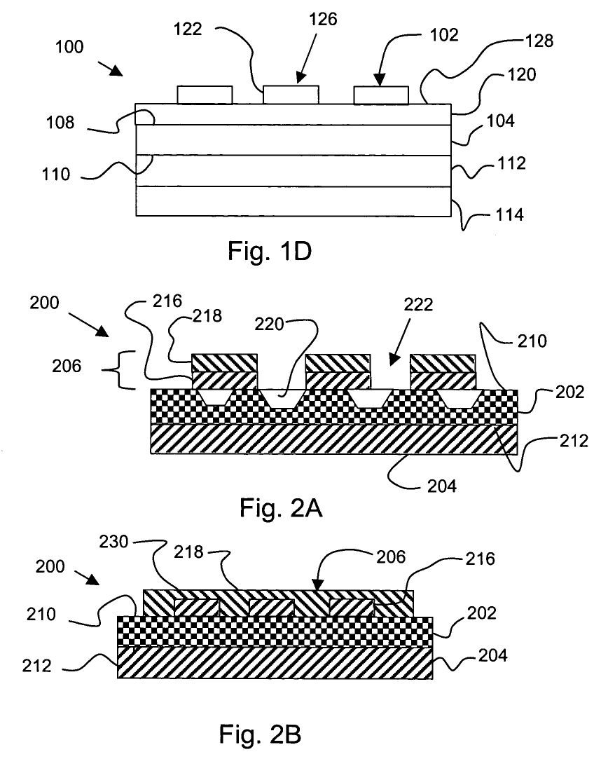

An article 100 comprising one embodiment according to the present invention is shown in FIG. 1A. The article 100 is a biological material detecting label, and includes a detecting system 102; a facestock 104 having an upper or first surface 108 and lower or second surface 110, wherein the detecting system 102 overlays at least a portion of the facestock film first surface 108; an adhesive layer 112 supported on the facestock film second surface 110; and, optionally, a release liner 114 releasably adhered to the adhesive layer 112. In the embodiment shown in FIG. 1A, the detecting system 102 includes an immunoassay device.

The detecting system 102 of the article 100 can have alternative detecting devices. Some of these alternative detecting devices include components with particular spatial orientations or physical arrangements as shown in FIGS. 1B-1D. The detecting device in FIG. 1B is an immunoassay device that includes an immobilized antibody layer 120 and a detector antibody layer 122. The immobilized antibody layer 120 is applied to, or printed in an array or pattern 124 on the upper surface 108 of the facestock 104. The detector antibody layer 122 is generally continuous and overlays the immobilized antibody layer 120 and at least a portion of the upper surface 108 of the facestock 104.

The detecting system 102 in FIG. 1C is an immunoassay device that includes the immobilized antibody image 120 applied to the upper surface 108 of facestock 104 in the array or pattern 124, and the detector antibody image 122, which overlays only the immobilized antibody image 120. The detector antibody layer 122 can be applied in register with the underlying immobilized antibody image 120 by, for example, flexographic, screen, and inkjet printing methods.

The detecting system 102 in FIG. 1D is an immunoassay device that includes the immobilized antibody layer 120 and the detector antibody layer 122. The immobilized antibody layer 120 is generally continuous and supported by upper surface of the facestock 104. The detector antibody layer 122 is applied to, or printed in an array or pattern 126 on, an upper surface 128 of the immobilized antibody layer 120.

The facestock film 104 can be a film formed from paper or a polymer. In an illustrated embodiment, the facestock film is a clear, flexible extruded polyethylene film that has been hot stretched to introduce a multi-axial orientation and annealed. Other suitable polymer films include those films formed from polyolefins (linear or branched), polyamides, polystyrenes, nylon, polyesters, polyester copolymers, polyurethanes, polysulfones, styrene-maleic anhydride copolymers, styrene-acrylonitrile copolymers, ionomers based on sodium or zinc salts of ethylene methacrylic acid, polymethyl methacrylates, cellulosics, acrylic polymers and copolymers, polycarbonates, polyacrylonitriles, and ethylene-vinyl acetate copolymers. Included in this group are the acrylates such as ethylene methacrylic acid, ethylene methyl acrylate, ethylene acrylic acid and ethylene ethyl acrylate. Also included in this group are polymers and copolymers of olefin monomers having, for example, 2 to about 12 carbon atoms, and in one embodiment, 2 to about 8 carbon atoms. These include the polymer of α-olefins having from 2 to about 4 carbon atoms per molecule. These include polyethylene, polypropylene, poly-1-butene, etc. Films prepared from blends of copolymers or blends of copolymers with homopolymers are also useful. The films may be extruded as monolayered films or multi-layered films. The films may be oriented films (single or multi-axial) or non-oriented films.

The facestock film may be an untreated film that is amenable to antibody immobilization by various mechanisms, e.g., adsorption. In a particular embodiment, the film is first ultrasonically cleaned and then dried. Alternatively, this film may be treated by first exposing the film to an electron discharge treatment at the surface, e.g., corona treatment, and then printing with a fluorescing antibody receptor. Subsequent to the printing step, the film may be dried or heated to immobilize the receptor. Various means of drying include the use of radiant heat, convected air and freeze-drying.

The adhesive layer 112 can be a pressure sensitive adhesive for use with film substrates. Suitable pressure sensitive adhesives include rubber based adhesives, acrylic adhesives, vinyl ether adhesives, silicone adhesives, and mixtures of two or more thereof. The pressure sensitive adhesives may be selected from hot-melt, solvent based or water based adhesives with reference to application specific criteria. Included are the pressure sensitive adhesive materials described in “Adhesion and Bond”, Encyclopedia of Polymer Science and Engineering, Vol. 1, pages 476-546, Interscience Publishers, 2nd Ed. 1985, the disclosure of which is hereby incorporated by reference. Some suitable pressure sensitive adhesive materials contain as a major constituent an adhesive polymer such as acrylic-type polymers; block copolymers; natural, reclaimed, or styrene-butadiene rubbers; tackified natural or synthetic rubbers; or random copolymers of ethylene and vinyl acetate, ethylene-vinyl-acrylic terpolymers, polyisobutylene, poly(vinyl ether), etc. Other materials may be included in the pressure sensitive adhesive such as tackifying resins, plasticizers, antioxidants, fillers, waxes, etc.

In one embodiment, the adhesive layer 112 comprises a heat-activatable adhesive or thermoplastic film material. These include polyolefins (linear or branched), polyamides, such as nylon, polyester copolymers, ionomers based on sodium or zinc salts of ethylene methacrylic acid, polyacrylonitriles, and ethylene-vinyl acetate copolymers. Included in this group are the acrylates such as ethylene methacrylic acid, ethylene methyl acrylate, ethylene acrylic acid and ethylene ethyl acrylate. Also, included in this group are polymers and copolymers of olefin monomers having, for example, 2 to about 12 carbon atoms, and in one embodiment, 2 to about 8 carbon atoms. These include the polymers of α-olefins having from 2 to about 4 carbon atoms per molecule, for example, polyethylene, polypropylene, poly-1-butene, etc.

Optionally, when an adhesive layer is present the removable release liner 114 may also be provided. In this embodiment, a pressure-sensitive adhesive is coated onto a release coated liner (paper or polymer). Thereafter, the adhesive coated liner is pressure laminated to the exposed surface of the polymeric film. The release liner 114 can subsequently be removed and the article 100 can be adhesively applied to a substrate.

With reference to the immunoassay device of the label, and particularly to the antibodies; the device includes at least two types of antibodies—immobilized (capture) antibodies and detector antibodies. The capture antibodies are adjusted to react in the presence of certain pathogens.

The capture antibodies are biologically active ligands characterized by their ability to recognize an epitope of the particular toxic substance being tested. An epitope is generally that part of an antigenic molecule to which a T-cell receptor responds, or a site on a large molecule against which an antibody will be produced and to which it will bind. These capture antibodies are selected from such materials as antibodies, aptamers, single stranded nucleic acid probes, lipids, natural receptors, lectins, carbohydrates, and proteins. The capture antibodies are immobilized on the facestock film surface, or just below the surface and in operational contact with an analyte. Several species of capture antibodies may be included within capture antibody layer of the immunoassay device so that multiple pathogens can be simultaneously detected using a single pathogen-detecting label. The corresponding species of detector antibodies are within the detector antibody layer. Antibodies that are suitable for use in the labels of the present invention include both monoclonal and polyclonal antibodies, and include antibodies that are commercially prepared and available, having been selected with reference to application specific criteria. The capture antibodies are immobilized to the facestock film.

Generally, according to some embodiments of the present invention, a coating containing detector antibodies forms a detector layer adjacent to a layer that contains the capture antibodies. As noted above, the detector antibody layer can be continuous and overlay the capture antibody layer and a portion of the facestock (FIG. 1B), can be printed in register above the capture antibody layer (FIG. 1C), or can form an array or pattern overlaying a continuous capture antibody layer (FIG. 1D). The coating has sufficient porosity to allow analyte molecules, such as antigens, to migrate through it to the detector antibodies and further through to the capture antibodies. Migration of antigens is driven by capillary action and can reach a state of equilibrium within a period influenced by the porosity of the coating, e.g. within a 72 hour period. If the antigen encounters a species of antibody that is specific to an epitope thereof, it will then bind to it forming a detector/antibody complex. Once bound thereto, the bound antigen/antibody complex becomes too large to migrate back through the coating due to the porous structure configuration of the coating relative to the size of the complex. This can aid in preventing undesirable, e.g., pathogenic, material from migrating back into the product being tested. Rather, the antigen/antibody complex migrates toward the corresponding species of immobilized capture antibodies supported on the facestock film surface. The immobilized antibodies layer is arranged on the facestock film surface in predetermined patterns of simple icons, words, and the like. When the particular species of bound antigen encounters a particular corresponding species of immobilized antibody specific to a separate and distinct epitope thereof, further binding occurs. Upon the antigen binding to the two antibodies, a visible shape that corresponds to the pattern formed by the immobilized antibodies layer emerges on the facestock film as a result of the binding, thereby producing a visual indication of the presence of the analyte. Because the presence of the analyte corresponds to a change in the packaged material, a logical assumption can be made that the packaged material has made the change for which it is being tested or monitored, e.g., spoilage.

In one embodiment, the antibody or aptamer is quantitatively sensitized so as to visually identify only those pathogens that have reached a concentration level deemed harmful to humans. One method of providing this sensitization is by including a scavenger antibody that is a biologically active ligand characterized as having a higher affinity for the particular toxic substance than the capture antibody. The scavenger antibody is provided in a sufficient amount to bind with the particular toxic substance up to and including a specific threshold concentration. In this manner, the capture antibody will be prevented from binding with a detector antibody until the concentration of the particular pathogenic material surpasses the specific threshold concentration. The pathogen detecting system visually reports only those instances where concentration levels exceed a pre-determined level.

The detector antibodies form a layer that is porous to allow analytes (e.g., bacteria and other pathogens) through to the capture antibodies. The detector antibodies can have a bead of color attached thereto. When bacteria, for example, enter the detector antibody layer, they attach themselves to the detector antibodies. Then they further attach to the capture antibodies. As more antibodies form this “lock-and-key” structure, more of the tag-along color beads are added to form an image. The image corresponds to the pattern of the immobilized antibodies that were preprinted on a surface of the facestock film. The labeled antibodies act as an ink so that the printed pattern can be seen. The pattern, and thus the image, can be of any configuration, including a logo, a symbol, a warning, and any other word or phrase. Upon appearance of the image, a consumer would be alerted to the presence of a particular analyte.

With reference to the colored beads, suitable beads include, for example, colored latex beads and UV fluorescing beads. Such labeled antibodies may be prepared by diluting latex beads in a solution such as phosphate-buffered saline and mixing the solution gently to suspend and distribute the latex beads in the solution. An antibody solution is added to the latex bead suspension. After addition of the antibody, the solution is gently mixed and incubated. At the completion of the incubation, the labeled antibodies are washed with phosphate-buffered saline, and the sensitivity and specificity of the labeled antibody preparation are tested.

As noted hereinabove, the beads can be formed into a visual indicator, or can be formed into a non-visual indicator, such as a UV responsive indicator. In the latter system, the detector antibodies can be conjugated with photoactive compounds capable of producing a visual cue in response to a particular type of light exposure, for example, ultraviolet light. It is also contemplated to form a scanning system that detects luminescent properties that are visualized upon binding of the pathogen and antibody. Suitable types of detector antibodies include, e.g., those conjugated with dyes to produce a visual cue upon binding of the antigen and antibody. These conjugated antibodies are referred to as chromogenic ligands. In this method of construction, biological materials are measured directly with a biologically active ligand, e.g., an antibody; an aptamer, that is, a double stranded DNA or single stranded RNA molecule that binds to specific molecular targets, such as a protein or a metabolite; nucleic acid probe; and the like that can induce a conformational change to produce a detectable cue, such as a visually observable cue.

In an alternative embodiment, the detecting system 102 includes a conductive ink forming a layer disposed adjacent to an immunoassay device. Electrical leads are attached to the conductive ink layer. As the antigens are bound to the antibodies, the conductive ink is removed from the conductive ink layer to increase the electrical resistance of the conductive ink layer. An increase in the electrical resistance can be monitored or sensed, or can cause an electrical signal to be generated.

An analyte detecting system of the present invention where the analyte is a pathogen can exhibit an active shelf life in excess of one year under normal operating conditions. This enhances the use of a pathogen detection system on products that are intended to be stored for about that period. Other immunoassay devices useful in the present invention, as well as methods of detecting biological materials, are described in U.S. Pat. Nos. 6,379,908; 6,376,204; and 6,051,388; U.S. Published Patent Application Nos. 2002/0045200 and 2002/0009811; and International Patent Publications WO 01/79840 and WO 01/79850.

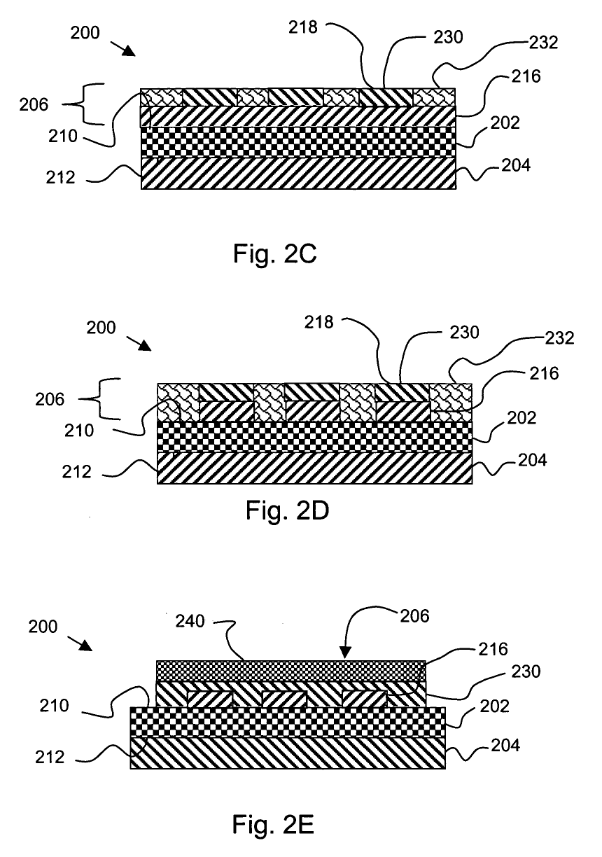

FIG. 2A shows an article comprising an embodiment of the present invention, which is a biological material detecting label 200. The label 200 of this embodiment is useful for detecting and indicating the condition of a material, such as the freshness of food, the absence of spoilage in food, the potency of medicine, and the like. The label 200 includes a film 202, an adhesive layer 204, and a detector system 206.

The film 202 is flexible, formed from a single or multiple thermoplastic layers, and has first and second major surfaces 210, 212. The film 202 can be formed from materials suitable to form the facestock film 104 shown in FIGS. 1A-1D, and can particularly be selected from substantially transparent polymer film materials to include polymers and copolymers such as polyolefin, polyacrylate, polystyrene, polyamide, polyvinyl alcohol, poly(alkylene acrylate), poly(ethylene vinyl alcohol), polyurethane, polyacrylonitrile, polyester, polyester copolymer, polycarbonate, cellulosics, polyacrylonitrile, alkylene-vinyl acetate copolymer, and the like. Particularly suitable films are BOPP, MYLAR, and polystyrene films. If the film 202 is a multi-layered film, it can comprise co-extruded and/or laminated layers of the same or a differing thermoplastic material.

The adhesive layer 204 is a pressure sensitive adhesive. Suitable pressure sensitive adhesives includes those disclosed hereinabove with reference to the adhesive layer 114 shown in FIGS. 1A-1D.

The detector system 206 is substantially the same as the detector system 102 described with reference to any one of FIGS. 1A-1D. Accordingly, it contains an immobilized capture antibody layer 216 and a detector antibody layer 218.

The film first surface 210 defines one or more micro-sized structures 220 arranged in a predetermined pattern 222. The micro-sized structures 220 are configured as channels, grooves, wells, and/or recesses having selected depths, widths and inner surface configurations and surface chemistries. Generally, the depths are less than the thickness of the film 202. The micro-sized structures 220 may be formed, for example, by an embossing process, such as those described in U.S. Pat. No. 6,200,399 and U.S. Published Patent Application No. 20010009172, the disclosures of which are hereby incorporated by reference. The structures 220 can facilitate contact between a vapor or liquid analyte and the detector system 206 by aiding in directing the analyte to the detector system 206. The inner surface chemistry can be determined, for example, by selection of material, by processing steps, by post-formation radiation treatment (e.g., corona treatment), and by post-formation chemical treatment.

With particular reference to suitable multi-layer films made of differing materials, co-extruded films can be used to provide a gradient of surface properties along the thickness of the structures or channels within the film. By way of example, a hydrophilic upper layer of a co-extruded film might hold a fluid sample while a lower layer having a more hydrophobic character might prevent flow out of the channels. In addition to gradients, defined boundary layers of differing properties can be utilized, particularly with reference to affecting the inner surface chemistry of the micro-structures 220.

With reference to FIG. 2B, another article comprising an embodiment of the present invention is shown. The article shown in FIG. 2B has many parts that are substantially the same as corresponding parts of the article 200 shown in FIG. 2A; the same reference numbers are used to identify such parts. The articles shown in FIGS. 2A and 2B differ at least in that the article shown in FIG. 2B includes an absorbent layer 230, and the micro-structures 220 are optional. The absorbent layer 230 can be incorporated into or can be the matrix that forms the layer that includes the detector antibodies 216. The absorbent layer 230 can increase the exposure of the antibodies 216, 218 to the analyte from the packaged material.

With reference to FIG. 2C, an article comprising an alternative embodiment according to the present invention is shown. The article is substantially similar to the label 200 shown in FIGS. 2A-2B. The label 200 shown in FIG. 2C differs from the label 200 of FIGS. 2A-2B at least in that the absorbent layer 230 includes a first absorbent layer 232 that contains the detector antibodies 216, and a second absorbent sublayer 234 that is disposed adjacent to the first absorbent sublayer 232, is generally initially free of detector antibodies 216, and overlays at least a portion of the layer that contains the immobilized antibodies 218.

In an alternative embodiment shown in FIG. 2D, the absorbent layer 230 of is adjacent to the both the layer containing the detecting antibodies 216, and the layer containing the capture antibodies 218. In this embodiment, the absorbent layer 230 is supported directly on the facestock film upper surface 210.

The absorbent material that comprises both of the absorbent layers 230, 232 can be a hydrophilic material, a selectively absorbent material, or a super-absorbent polymer. Examples of such super-absorbent polymers and polymer films are described in U.S. Pat. Nos. 6,403,700; 6,395,830; 6,251,479; 6,143,821; 6,033,769; and 6,051,317 and International Patent Publication WO 96/25958, the entire disclosures of which are hereby incorporated by reference. In alternative embodiments, the absorbent material can be formed from a natural, modified or substituted starch. Examples of useful starches include starch ester, starch ether and starch maleate, such as those described in U.S. Pat. No. 6,063,914, the entire disclosure of which is hereby incorporated by reference.

Suitable hydrophilic materials bind or absorb water, and include hygroscopic materials. For example, a suitable hydrophilic material is a polyvinyl polymer. Suitable alternative hydrophilic polymers can be formed from polyacrylic acid, polyacrylic acid copolymer, methacrylic acid, maleic acid, crotonic acid, and carboxylated sodium polyacrylate may be used. Additional hydrophilic materials include hydrophilic silicone-organic copolymer elastomers, such as those described in U.S. Pat. Nos. 4,851,216; 4,833,218; and 4,600,751, the entire disclosures of which are hereby incorporated by reference. Hygroscopic materials such as activated alumina, calcium chloride and silica gel may be used. In one embodiment, a crosslinkable (reactive) component is used in the absorbent layer. Examples of crosslinkable materials are those that contain carboxyl groups, hydroxyl groups or other functional groups that will react with a cross-linking agent.

With reference to FIG. 2E, the article 200 is substantially the same as the article 200 shown in FIGS. 2A-2D, except that a permeable, pigmented layer 240 is supported on the detector device 206. The entrance of the analyte into the detector device 206 allows the analyte to attach to the capture antibodies 218. The pigmented layer 240 can include a permeable, polymeric layer having pigment particles incorporated within the polymeric structure. The permeable layer can be a woven or a non-woven polymeric film. Useful polymeric films can be formed from the same or similar materials as the facestock film 104 described hereinabove. In one embodiment, the permeable layer is permeable to gases, but not liquid or vapor. In another embodiment, the permeable layer is permeable to vapor, but not liquid or gas. In yet another embodiment, the permeable layer is permeable to liquid, but not vapor or gas. Alternatively, the pigmented layer 240 has an inward facing major surface and an outward facing major surface. Either of the surfaces can support a non-pigmented sublayer and an ink sublayer adjacent to the non-pigmented sublayer (not shown). In one embodiment, the ink sublayer is printed on the non-pigmented sublayer and disposed or sandwiched between the non-pigmented layer and the detector device 206. The pigmented layer 240 increases the visual contrast of a visible indication caused by the presence of the analyte and the interaction of the analyte, the detector antibodies and the capture antibodies.

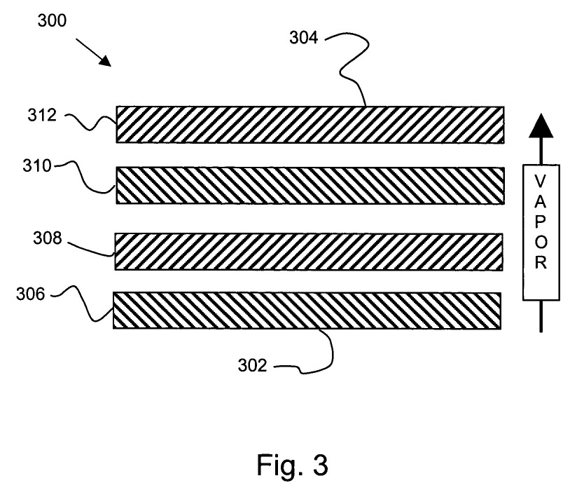

With reference to FIG. 3, an article 300 comprising another embodiment according to the present invention is shown. The article 300 includes a multi-layered structure having a first surface 302 and a second surface 306. The multi-layered structure includes a film substrate 306, a selectively permeable layer 308, an analyte detector layer 310 and a backing layer 312. The first surface 302 is generally an inward facing surface and is oriented toward a packaged material that contains or will produce an analyte, such as a vapor indicating spoilage of the packaged material. The article second surface 304 is generally an outward facing surface oriented away from the material to be tested or monitored. In clear packaging, the article 300 can be oriented such that the article second surface 304 faces toward the packaging inner sidewall and is thus visible through the clear packaging material to an observer who is outside of the package looking in. According, the article first surface 302 is oriented toward the packaged material that it is monitoring for an expected change in condition, e.g., spoilage.

The film substrate 302 is porous or perforated and can be formed from non-biodegradable materials, or materials with reduced biodegradability. In one embodiment, the film substrate 302 is a clear, flexible perforate polyethylene film.

The selectively permeable layer 308 can be selected to allow only vapor through, to the exclusion of liquid. In alternative embodiments, the selective permeability can allow a particular analyte through to the exclusion of interfering species. In yet other alternative embodiments, the selectively permeable layer 308 allows only uni-directional flow, or is hydrophilic or hygroscopic and encourages wicking or transport of the analyte to the detector layer 310.

The detector layer 310 in one embodiment includes at least one dye deposited onto the backing layer 312 in a predetermined pattern or array. The dye has a distinct and direct spectral absorbance or reflectance response to a particular analyte, for example, a distinct biological material.

In another embodiment, the detector layer 310 includes a combination of at least a first dye and a second dye deposited onto the backing layer 312 in a predetermined pattern combination. The first and second dyes may be selected from porphyrin, chlorin, chlorophyll, phthalocyanine and salen, and their metal complexes. This detector device is particularly useful in detecting metal ligating vapors. U.S. Pat. No. 6,368,558, which is incorporated in its entirety by reference, discloses Artificial noses and Artificial tongues that incorporate patterns of porphyrin and metalloporphin dyes.

In an alternative embodiment, the backing layer 312 and the substrate film 306 can seal along a continuous peripheral edge to enclose the semi permeable layer 308 and the detector layer 310. The backing layer 312 and the substrate film 306 cooperate with each other to allow the analyte in and to keep any indicator molecules in. In one embodiment, a barrier layer or coating (not shown) that is permeable to, for example, food spoilage products but not to the indicator molecule is disposed on inner surfaces of backing layer 312 and the substrate film 306 to form a continuous envelope.

The backing layer 312 can be formed from any of the materials suitable for use in forming the facestock film 104 shown in FIG. 1A. The backing layer 312 can optionally have a clear adhesive layer (not shown) applied to the outward facing surface. Thus, some of the articles in accordance with the present invention may be adhered to the inside of a clear flexible food storage bag or to the inside of a rigid food storage container by the adhesive layer.

The detector layer 310 can include a metal-complex containing sensor responsive to a gas or to a vapor. Specifically, the detector layer 310 can be a metal coordinated complex immobilized as an unsupported layer or as a coating on a substrate. The layer or coating can be formed by printing, casting, roller application, brushing, spraying, and the like. Suitable metals to form the complex include palladium, platinum, ruthenium, iron, copper, nickel, zinc, gold, rare earth metals, cobalt, iridium, titanium and vanadium. Suitable ligands include fluorescein or FLUOREXON, which is commercially available from Sigma-Aldrich, Inc. (St. Louis, Mo.). FLUOREXON can react with Na₂(PdCl₄) to yield a Pd-FLUOREXON complex suitable for use with this embodiment. Other useful palladium complexes include palladium dializarin red, (Nbu₄)₂[PdAlzarin₂] and the palladium complex of alizarin complexone. Additional suitable complexes include those that include a dye, a complexone, a Schiff base, or a rare earth polyamino carboxylate.

During use, the complex releases a detectable component in response to a preferential binding of a vapor to the metal of the complex. The vapor includes the analyte, for example, food spoilage products such as sulfur-, nitrogen-, alcohol-, carbonyl- and phosphorus-containing substances. The detectable component may comprise a fluorophore or a chromophore. Because the complex is pre-arranged in a pattern or array, and the the detectable component is released but remains generally proximate to the former complex, the detectable component can then form an image or word in a pattern or array that corresponds to the pre-arranged pattern or array of the complex. The corresponding pattern or array is detectable through the backing layer 312 and further detectable through any proximate clear packaging material.

During use and in general, certain substances come into contact with the detector layer 310 to cause a color change and alert the user to the presence of undesirable material or an undesirable condition. More specifically, the analyte in liquid, solid or vapor form can move through the pores or perforations in the film substrate 306. From there, vapors containing the analyte can move through the selectively permeable layer 308 in the direction indicated by the directional arrow labeled VAPOR. The analyte-containing vapor contacts the detector layer 310, which responds to the contact by changing color. Because the color-changing complex is pre-arranged in a pattern or array, the changing color can create an image or word to indicate that the reaction has happened. Thus, it is visibly deducible that the generally undesirable change has occurred, for example, the packaged meat has spoiled.

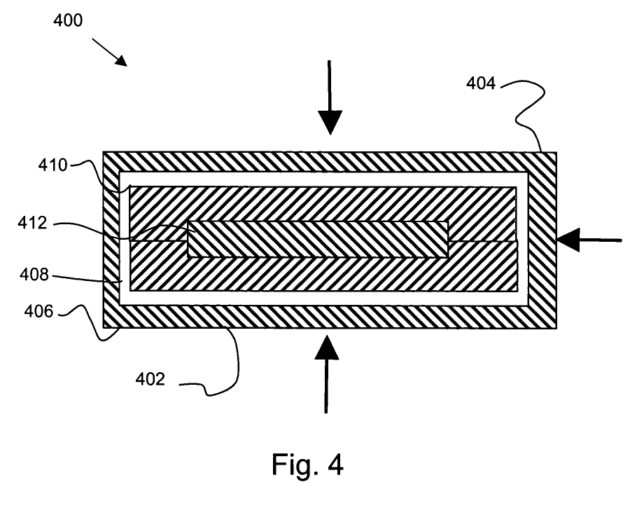

With reference to FIG. 4, an analyte detecting system comprising an embodiment according to the present invention is shown. The system includes a packet 400 that is is useful for detecting toxins under conditions other than those related to food storage.

The packet 400 includes an outer envelope 402 having a defined front surface 404 and rear surface 406. The envelope defines a cavity 403, and disposed within the cavity is an intermediate layer 410. Disposed within the intermediate layer 410 is a detector structure 412.

The envelope 402 in one embodiment is formed from a heat-sealable polymer material and can be selected from the list of materials disclosed herein as suitable for use as the facestock film 104 shown in FIG. 1A. The envelope 402 is generally transparent or has transparent portions and is porous, permeable or perforate to allow the movement of liquid and vapors therethrough. In one embodiment, the envelope 402 is formed by heat-sealing, or otherwise adhesively sealing, the peripheral edges of two generally planar sheets or films together.

In this embodiment, the intermediate layer 410 includes a hydrophilic binder, an indicator dye and alumina particles. The binder and the alumina particles can be arranged as a single layer or as adjacent layers (distinct or gradient layers) with the indicator dye dispersed throughout the layer. A suitable hydrophilic binder is disclosed in U.S. Pat. No. 6,653,427, which is hereby incorporated by reference in its entirety. In particular, a suitable binder is a polyHEMA copolymer polyalcohol binder that provides a porous vehicle for the indicator dye and the osmotic control agents. Suitable amounts are in ranges of: for the binder about 10 weight percent to about 90 weight percent, for the indicator dye about 0.01 weight percent to about 5 weight percent, and for the alumina particles about 10 weight percent to about 90 weight percent. Amount selections can be made with reference to performance criteria to include solubility, application ease, flexibility, transparency, and the like.

The indicator dye can be a polyazamacrocyclic transition metal complex. Platinum complexes are also useful in the present embodiment. The polyazamacrocyclic transition metal complex undergoes a detectable color change upon exposure to, for example, a biogenic amine. Biogenic amines include putrecine (1,4-diaminopentane), cadaverine (1,5-diaminopentane) and histamine (5-imadazole-ethylamine). The polymer film or polymeric coating is applied to a surface of the label and is exposed to vapors and/or liquids that can potentially contain the analyte. A suitable sensor system is described in International Publication WO01/77667, and a suitable dye indicator is disclosed in International Publication WO00/13009, the disclosures of which are hereby incorporated by reference in their entirety. A particularly useful coating weight for the detecting layer is in a range of from about 10 to about 60 grams per square meter (gsm).

The alumina particles are the osmotic control and permeation modulator. The alumina particles are nano-sized or nano-scale, meaning they have an average or mean diameter in a range of from about 0.01 nanometers (nm) to about 1000 nanometers. In one embodiment, the average or mean diameter is in a range of from about 1 nanometer to about 10 nanometers, and in another embodiment the average or mean diameter is in a range of from about 100 nanometers to about 200 nanometers. In another embodiment according to the invention, the alumina particles have an average particle size diameter below 100 nanometers. The preferred range is in from 20 to 60 nanometers. Suitable nanoparticles include DISPAL 23N4-80 and DISPAL 18N4-80 from Sasol North America Inc. The nanoparticles can be in either powder form or in a water dispersible boehmite alumina system. For a liquid boehmite alumina system, DISPAL 23N4-80 contains 80% aluminum oxide; the primary particle size of the alumina is 50 nm, and the dispersed particle size is 90 nm. DISPAL 18N4-80, also contains 80% aluminum oxide and the primary particle size of the alumina is 50 nm, however, its dispersed particle size is 110 nm. Other useful alumina sols are available from Nissan Chemical Industries under the names of ALUMINASOL 100 and ALUMINASOL 200. Other inert inorganic nanoparticles that can be used include TiO₂, SiO₂, and the like. Suitable colloidal silicas are available commercially from Nissan Chemical Industries under the names of SNOWTEX ST-PS—S, SNOWTEX ST-UP, and the like, and from Dupont Specialty Chemicals, Inc. (Wilmington, Del.) under the names of LUDOX CL and LUDOX AM; or from Grace Davison, Inc. (Columbia, S.C.) under the name of SYLOJET 400P.

In alternative embodiments, the particles are formed from differing metals or from ceramic or vitreous materials. The particles in the layer controllably form tortuous paths having predetermined dimensions. By controlling, for example, the size, type and concentration of the particles in the intermediate layer 410, the osmotic flow rate, selectivity, and/or permeation moderation can be influenced and controlled. The particle size (average), the particle size distribution, and the composition of the particles are factors that can be selected to control the performance of the intermediate layer with reference to, for example, osmotic flow rate, selectivity, and/or permeation moderation. Suitable alumina particles are commercially available as, for example, DISPAL 23N4-80 and DISPAL 18N4-80 from Sasol North America Inc. (Tucson, Ariz.). In addition to alumina, titanium dioxide, silicon dioxide and other like inorganics can be interchangeable with reference to the parameters described herein.

Alternatively, the intermediate layer 410 can be formed from, for example, fiber (woven fabric or non-woven mat), porous films, and perforate films. A suitable material for use in the intermediate layer 410 is polytetraflouroethylene (PTFE) fibers. In one embodiment, the intermediate layer 410 is permeable to vapors, but not to liquid.

An adhesive layer (not shown) can be a clear/transparent pressure sensitive adhesive and can adhere the packet 400 to an inner wall of a container. A particularly useful coat weight for the adhesive layer is in a range of from about 15 to about 25 gsm. The packet 400 can thus be positioned and maintained in a viewable orientation relative to an observer.

In an alternative embodiment, the packet 400 can be adhesively mounted on the outside of a packaging material, provided that the packaging material has sufficient vapor permeability, or gas permeability if a gas is be analyzed rather than a vapor. If adhered to the outer surface of the gas or vapor permeable packaging, the adhesive must either be discontinuous or vapor or gas, respectively, permeable itself. Accordingly, the adhesive material, the method of application, and/or the pattern in which the adhesive is applied must be selected with reference to application specific criteria.

In an alternative embodiment, the package-facing surface of the packet 400 can have a single sharp projection or a plurality of such projections that score or perforate the packaging material, the adhesive creates a seal over the perforation to maintain the sealing integrity of the packaging. The projections can alternatively be hollow and the liquid, gas or vapor path between the packaged material and the detector layer 412 is continuous through the hollow projections. The hollow pathway through the projections can be further modified to be selective in the material that it transports. This selectivity can be achieved by, for example, using a polar material to form the projections, controlling the diameter of the aperture defined by the projections, packing the projections with a selectively permeable material, arranging the projections to be or to not be in contact with, for example, liquid contents or vapor contents by physical placement, and the like.

During use, the vapors travel through the envelope 402 and then through the intermediate layer 410 in the direction indicated by the directional arrows to contact the detector 412. In response to the contact with an analyte of interest in the vapors, the detector 412 changes appearance, for example, changes color or changes from colorless to colored. The change indicates that the analyte is present, and can further indicate at least an approximation of the concentration of the analyte.

With reference to the analyte, the packet 400 can be used to detect, for example, the presence of toxins and pathogens in medicines, blood products, or medical samples. Alternatively, the packet 400 can be used in the field of medical analysis and diagnostics. Analytes that are contemplated as being detectable by the packet 400 include, but are not limited to, the by-products or organisms of E. coli, ciguatoxin, salmonella, botulism, listeria, scrape (“mad cow disease”), campylobacter, shigella, cyclospora, anthrax, streptococus Group A antigen, streptococus Group B antigen, viral antigens, antigens associated with autoimmune disease, allergens, tumor antigens, HIV I or HIV II antigen, antigens specific to hepatitis, or host response (antibodies) to these and other viruses and viral material. Also included as a detectable material are those selected from biologically active materials, such as enzyme, hormone, saccharide, protein, peptide, lipid, carbohydrate, nucleic acid, and hapten. In addition, other classes of material are included as being detectable to include phytochemicals, nutraceuticals, drugs of abuse, and therapeutic drugs. Non-biological materials may also be include as detectable, such as explosives, pesticides, solvents, inks and dyes, pigments, herbicides, and the like.

The detector 412 can be relatively quick in response time between exposure and indication, have improved color contrast, have a selectable color scheme, have improved UV fluorescence, have a reduced migration of dye from the detector 412 out of the package 400, and have an improved hydrophilicity without an increased water solubility.

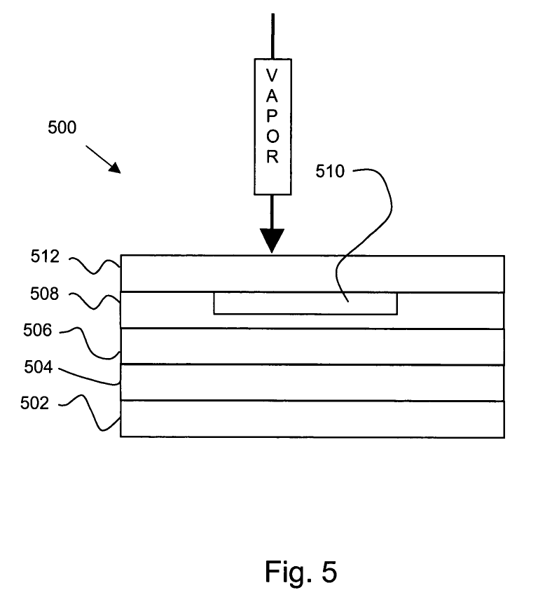

With reference to FIG. 5, a label 500 according to the present invention is shown. The label 500 includes a release layer 502, a first adhesive layer 504 supported by the release layer 502, a facestock layer 506 adjacent to the first adhesive layer 504 and opposite the release layer 502, a second adhesive layer 508 overlaying the facestock layer 506 opposite the first adhesive layer 504, a detecting layer 510 overlaying a portion of the second adhesive layer 508 opposite the facestock layer 506, and a backing layer 512 overlaying the second adhesive layer 508 and the detecting layer 510.

The release layer 502 is a silicone coated cellulosic release liner. In alternative embodiments, suitable commercially available release liners are interchangeable with reference to application specific criteria.

The first adhesive layer 504 is a pressure sensitive adhesive and is transparent. Suitable thicknesses for the adhesive layer 504 include those thicknesses in a range of from 10 to 50 gsm. After the release layer 502 is removed from a surface of the first adhesive layer 504, the label 500 can be adhered to an inner surface of a package container. That is, an observer from outside the package container would see the first adhesive layer 504 and the underlaying facestock layer 506 therethrough. Suitable alternative adhesive materials are listed with reference to the adhesive layer 112 shown in FIG. 1.

The facestock layer 506 is a transparent, partially transparent, or transparent to preselected radiation types (e.g., ultraviolet radiation, or fluorescence) or is transparent in portions and opaque in other portions. The facestock layer 504 is an extruded, stretched polyethylene film. Suitable alternative materials for producing the facestock layer 504 include materials listed as suitable for use forming the facestock film 104 shown in FIG. 1.

The second adhesive layer 508 is a transparent UV curable adhesive formed from a polyacrylate. In alternative embodiments, suitable adhesive materials can be selected from the adhesive materials listed as suitable for the adhesive layer 35 USC § 112 shown in FIG. 1.

In alternative embodiments, the second adhesive layer 508 is discontinuous and does not underlay the detecting system 510, that is, no adhesive material is sandwiched between the detecting system 510 and the facestock layer 506. In such an alternative embodiment, the adhesive material can contain opacifying fillers or ingredients, or can be otherwise opaque as it would not interfere with observation of the detecting layer 510 from outside of the packaging container. The alternative adhesive layer can retain the detecting layer 510 against the facestock layer 506 by overlaying the detecting layer 510, rather than underlaying. In yet another alternative embodiment, the detecting system 510 is printed onto, and self adheres to, the facestock layer 506.

The detecting system 510 is substantially similar to the detecting system 412 shown in FIG. 4. Thus, it includes osmotic controls, indicator dyes and a hydrophilic binder. In alternative embodiments, the detecting system 510 is substantially similar to the detecting system 102 shown in FIG. 1. The detecting system 510 is screen or flexographically printed onto the facestock layer 506 and dried at a temperature in a range of from about 40 degree Celsius to about 100 degree Celsius. The dry time and temperature can be adjusted to accommodate variables such as coating thickness, solids content, relative humidity, print or coating method, solution composition, and the like.

The backing layer 512 is a nonwoven, vapor-permeable polymeric fabric or mat. A suitable commercially available nonwoven material is TYVEK from DuPont, Inc. (Wilmington, Del.). In this embodiment, the backing layer 512 is white to increase the visible contrast with the detecting layer 510. The backing layer 512 is permanently adhered to the facestock layer 506 by the second adhesive layer 508.

During use, vapors from the monitored material, for example, fruit, flow toward the backing layer 512 as indicated by the directional arrow labeled VAPOR. While liquid is turned back, the vapor continues through the backing layer 512 and into the detecting layer 510. The backing layer osmotic flow control regulates the flow of the analyte, which may be contained in the vapors, and the flow is facilitated by the hydrophilic nature of the binder. When the vapor contains the analyte, or a sufficient quantity of the analyte, the indicator dye in the detecting layer indicates that the analyte is present. This can be a color change. The color change is enhanced by the white background provide for by the backing layer 512. The change signals that the analyte is present and can indicate, depending on the application, that the food is not fresh, the fruit is ripe, the meat is spoiled, the medicine is not potent, the solvent has leaked, etc.

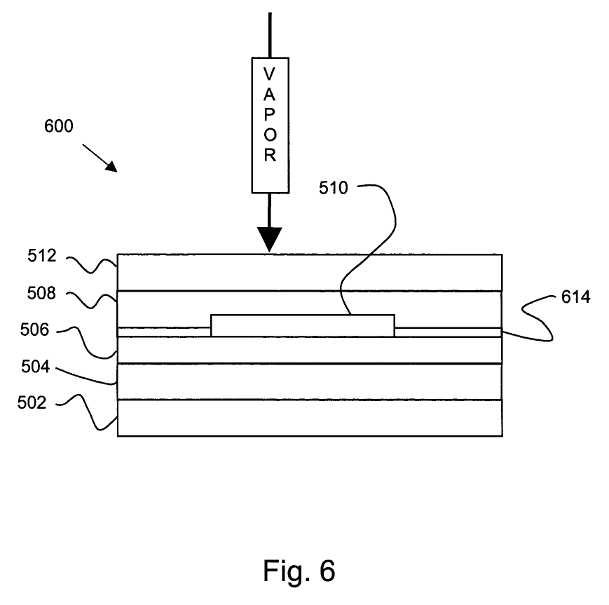

A label 600 comprising another embodiment of the present invention is shown in FIG. 6. The label 600 is substantially similar to the label 500 shown in FIG. 5, and has many parts that are substantially the same as corresponding parts shown in FIG. 5; accordingly, the same reference numbers are used to indicate such corresponding parts. The label 600 of the present embodiment differs in that it includes a print layer 614, and the detecting layer 510 is printed directly to a surface of the facestock layer 606.

During use, the print layer 614 defines the shape, word, icon or other that the signaling detecting layer 510 uses to indicate the presence of the analyte. The print layer 614 can further be phrases such as warnings or instructions. Additionally, the detecting layer 510 can be one of a plurality of differing detecting systems, each detecting system capable of sensing a different analyte. The print layer 610 can help to identify which of the differing analytes is being sensed and indicated by a corresponding detecting layer 510.

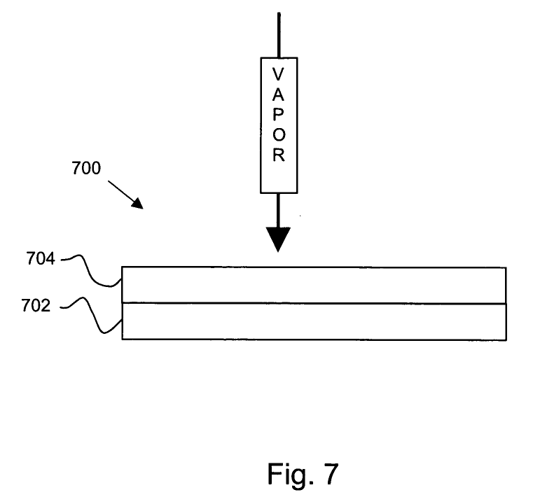

With reference to FIG. 7, a label 700 comprising another embodiment of the present invention is shown. The label 700 includes a facestock film 702 and a detecting layer 704. The label 700 can sense whether an analyte is present in a flow of a vapor contacting the detecting layer 704. The vapor flow is indicated by the directional arrow labeled VAPOR.

The facestock film 702 is substantially similar to the facestock 104 shown in FIG. 1. The detecting layer 704 is a layer of material formed according Example 1, hereinbelow.

### EXAMPLES

### Example 1

### Producing a Detecting Layer

Procedure.

In Example 1, a coating material is formed by mixing 25 grams of Alumina nano-sized particles, 0.3 grams of Pd-F complex, 25 grams of polyHEMA copolymer solution, and 49.7 grams of water together in a mixer. The amounts can be changed for result effective reasons. For example, more water could be added to reduce the viscosity of the coating material, more Pd-F complex could be added to increase the visibility/detectability of the label's indication, more binder could be added to increase the hydrophilicity of the resultant detecting system, and more alumina could be added to decrease the flow rate through the resultant detecting system. Alternative, by using an alumina particle having a larger or smaller diameter, the flow rate or osmotic flow can be controlled, and by using a binder with an increased or decreased hydrophilicity, the hydrophilicity of the resultant detecting system can be controlled.

The coating material is a substantially homogeneous solution or slurry and can be applied to a substrate as, for example, a printing ink. Accordingly, suitable application methods include ink jet, spray, metering, brush, roller, doctor blade, and the like.

The coating material is applied to a facestock surface in a predetermined pattern and wet coat thickness. The pattern can be a symbol, a shape, letters, numbers and/or words. The coating material is allowed to dry and sticks to the facestock surface. The thickness is about 25 micrometers thick (wet), and after drying is less than about 25 micrometers thick (dry). The composition of the dry material is 76 percent by weight alumina particles, 0.9 percent by weight Pd-F complex, and 22.9 percent by weight binder.

### Example 2

### Producing a Binder

Binders suitable for use in an embodiment according to the present invention are produced in EXAMPLES 2(a)-2(g). EXAMPLE 2(e) is described in detail below, the remaining examples are produced in the same manner as EXAMPLE 2(e) except for differing ratios of acrylate material as described in TABLE 2. EXAMPLE 2(e) is produced by mixing the ingredients listed in TABLE 1 according to the following procedure.

TABLE 1
Binder formulary.

| | Weight (grams) |
|---|---|
| Reactor Charge | |
| 2-Hydroxyethyl Methacrylate | 200 |
| 4-Hydroxybutyl acrylate | 100 |
| Ethanol | 400 |
| Deionized Water | 260 |
| Initiator Charge | |
| Deionized Water | 10 |
| Sodium Persulfate (0.5%) | 1.5 |
| Cook-Off Initiator #1 | |
| Deionized Water | 10 |
| Sodium Persulfate | 0.3 |
| Cook off Initiator #2 | |
| Deionized Water | 10 |
| Sodium Persulfate | 0.3 |
| Cook off Initiator #3 | |
| Deionized Water | 10 |
| Sodium Persulfate | 0.3 |

Procedure.

The Reactor Charge is weighed out into a flask and poured into a reaction kettle with mixing, and is heated with an 80° C. jacket and a nitrogen purge kettle. The Cook-Off initiator #1 is weighed into a small beaker and mixed until the solids dissolve. About three hours after the addition of the Reactor Charge, Cook-Off initiator #1 is added to the kettle. The Cook-Off initiator #2 is weighed into a small beaker and mixed until the solids dissolve. About one hour after the addition of Cook-Off initiator #1, Cook-Off initiator #2 is added to the kettle. The Cook-Off initiator #3 is weighed into a small beaker and mixed until the solids dissolve. One hour after the addition of Cook-Off initiator #2, Cook-Off initiator #3 is added to the kettle. About one-half hour after the addition of Cook-Off initiator #3, the kettle contents are cooled and discharged.

Results.

The products synthesized in EXAMPLES 2(a)-2(g) exhibit the following general properties: The appearance is a clear to light-yellow gel-free liquid. The solids content is 30%. The average level of residual monomers is about: 0.02 for 2-Hydroxyethyl Methacrylate, and <0.01 weight percent for 4-Hydroxybutyl Acrylate.

TABLE 2
Results for EXAMPLES 2(a)-2(g)

| | Ratio | | Tensile | Testing | Stress at | Young's | |
|---|---|---|---|---|---|---|---|
| | HEMA | | % Strain | Stress at | 2% yield | Modulus | MVTR |
| EXAMPLE | Copolymer | Tg | at Peak | Peak, (psi) | (psi) | (psi) | g/m²/day |
| 2(a) | 100/0 | 85° C. | brittle | | | | 2110 |
| 2(b) | 80/20 | 77° C. | 7.1 | 3,385 | 2,311 | 130,125 | 2350 |
| 2(c) | 75/25 | 54° C. | 8.3 | 2,058 | 1,609 | 69,536 | 2590 |
| 2(d) | 70/30 | 47° C. | 9.2 | 1,529 | 976 | 51,471 | 2480 |
| 2(e) | 67/33 | 34° C. | 12.3 | 1,162 | 793 | 26,816 | 2540 |
| 2(f) | 60/40 | 22° C. | 337 | 805 | 114 | 13,647 | 2640 |
| 2(g) | 50/50 | 14° C. | 607 | 165 | 15 | 4,139 | 3180 |

### Example 3

### Alternative Formulations Using Titanium Dioxide Particulate

Preparation:

In EXAMPLE 3(a), AIRFLEX 300 (having a pH of 6.6) is weighed out at 1500 grams (g). AIRFLEX 300 is commercially available from Air Products and Chemicals, Inc. (Allentown, Pa.) and is 55 percent by weight solids, having a pH of between about 6.6, and further having a glass transition temperature (T_{g}) of about 17° C. To the AIRFLEX 300, 600 grams of titanium dioxide (TiO₂) (wet) and 900 grams of a Palladium-FLUOREXON (Pd-F) complex solution are added. The Pd-F solution is 0.99 weight percent active.

In Example 3(b), 1000.9 g KRONOS 4102 (72% solids by weight) is added to 1000.1 g of the binder used in Example 2(d) (i.e., a 30% ratio PolyHEMA copolymer). KRONOS 4102 is a titanium dioxide slurry commercially available from Kronos, Inc. (Houston, Tex.). To that mixture is added 600 g of the Pd-F solution used in Example 3(a).

In both Examples 3(a)-3(b), the materials are mixed until uniform. The uniform mixtures are applied to a surface by screening or flexographic printing. The wet coating layer is dried to form a detecting layer. The detecting layer results are shown in TABLES 3-4.

Results:

TABLE 3
Results for Example 3(a)

| | Weight | Wt % | Wt % |
|---|---|---|---|
| | (g) | on wet | on dry |
| Binder | 1500.0 | 27.50 | 63.25 |
| TiO₂ | 600.0 | 15.68 | 36.07 |
| Pd-F complex | 900.0 | 0.30 | 0.68 |
| solution | | | |
| Total weight (wet) | 3000.0 | | |
| Total weight (dry) | 1304.3 | | 100.00 |
| solid % | | 43.5 | |

TABLE 4
Results for Example 3(b)

| | Weight | Wt % | Wt % |
|---|---|---|---|
| | (g) | on wet | on dry |
| Binder | 900.0 | 27.00 | 70.13 |
| TiO₂ | 900.0 | 11.25 | 29.22 |
| Pd-F complex | 600.0 | 0.25 | 0.64 |
| solution | | | |
| Total weight (wet) | 2400.0 | | |
| Total weight (dry) | 923.9 | | 100.00 |
| solid % | | 38.5 | |

The processes and embodiments described herein are examples of structures, systems and methods having elements corresponding to the elements of the invention recited in the claims. This written description may enable those skilled in the art to make and use embodiments having alternative elements that likewise correspond to the elements of the invention recited in the claims. The intended scope of the invention thus includes other structures, systems and methods that do not differ from the literal language of the claims, and further includes other structures, systems and methods with insubstantial differences from the literal language of the claims.

What is claimed is:

1. An analyte detecting article for mounting on a surface of a packaging substrate and for detecting an analyte, comprising:
a facestock film having first and second surfaces;
an adhesive layer having first and second surfaces, and the adhesive layer first surface is adhered to the facestock film second surface, and the adhesive layer second surface being operable to adhere to the packaging substrate surface; and
a detecting system adjacent to the facestock film, and the detecting system is responsive to contact with the analyte by indicating that such contact has occurred.

2. The article of claim 1 wherein the detecting system comprises an immunoassay device.

3. The article of claim 2 wherein the immunoassay device comprises:
a capture antibody layer comprising a species of capture antibodies, which are applied in a predetermined pattern on the upper surface of the facestock film;
a porous detector antibody layer, comprising a species of detector antibodies corresponding to the capture antibodies, the detector antibody layer overlying the capture antibody layer, whereby simultaneous binding of one or more of the capture antibodies and one or more of the corresponding detector antibodies with the analyte results in a visual signal.

4. The article of claim 3 wherein the detector antibodies comprise a chromogenic ligand.

5. The article of claim 1 wherein the detecting system comprises a metal-complex containing sensor.

6. The article of claim 5 wherein the metal-complex containing sensor comprises a polyazamacrocyclic transition metal complex.

7. The article of claim 1 wherein the detecting system comprises at least one dye deposited onto the facestock in a predetermined pattern, wherein the dye has a distinct and direct spectral absorbance or reflectance determined by the presence or absence of the analyte, and wherein the dye is selected from the group consisting of porphin, chlorin, chlorophyll, phthalocyanine and salen, and their metal complexes.

8. The article of claim 7 wherein the upper surface of the facestock film defines microstructures configured to facilitate the transport of liquid or vapor to the detecting system.

9. The article of claim 1 wherein the adhesive layer is a pressure sensitive adhesive.

10. The article of claim 9 further comprising a release layer adjacent to the adhesive layer second surface.

11. The article of claim 1 further comprising an absorbent layer adjacent to the detecting system and supported by the facestock film first surface.

12. The article of claim 11 wherein the absorbent layer comprises an absorbent material selected from super-absorbent polymers, starch, and polyvinyl polymers, silicone-organic copolymer elastomers, activated alumina, calcium carbonate and silica gel.

13. The article of claim 1 wherein the facestock film is flexible and transparent.

14. The article of claim 3 further comprising a pigmented layer overlying the immunoassay device, the pigmented layer being operable to increase the contrast of an image formed in response to the analyte contacting the capture antibodies.

15. The article of claim 14 wherein the pigmented layer comprises a non-woven polymeric layer.

16. The article of claim 3 wherein the analyte is a predetermined pathogen, and the detector antibody layer further comprises a scavenger antibody having a higher affinity for the predetermined pathogen than for the capture antibodies, and the scavenger antibody is present in a sufficient amount to bind with the analyte up to and including a specific threshold concentration.

17. The article of claim 3 wherein the capture antibody layer is continuous or is in register with the detector antibody layer.

18. The article of claim 1 wherein the adhesive layer is permeable to the analyte.

19. The article of claim 1 wherein the adhesive layer is discontinuous to allow the analyte to move therethrough.

20. The article of claim 1 wherein the packaging substrate surface is an inner sidewall surface, and the detecting system is visible or detectable through the packaging substrate.

21. The article of claim 1 wherein the facestock film defines hollow protrusions that extend through the packaging substrate to define a path configured to allow the analyte to move from a monitored material to the detecting system.

22. The article of claim 1 wherein the analyte is selected from the group consisting of by-products or organisms of E. coli, ciguatoxin, salmonella, botulism, listeria, scrape (“mad cow disease”), campylobacter, shigella, cyclospora, anthrax, streptococus Group A antigen, streptococus Group B antigen, viral antigens, antigens associated with autoimmune disease, allergens, tumor antigens, HIV I or HIV II antigen, antigens specific to hepatitis, and corresponding antibodies thereof.

23. The article of claim 1 wherein the analyte is selected from the group consisting of biogenic amines, enzyme, hormone, saccharide, protein, peptide, lipid, carbohydrate, nucleic acid, hapten, phytochemicals, nutraceuticals, drugs of abuse, and therapeutic drugs.

24. The article of claim 1 wherein the analyte is selected from the group consisting of explosives, pesticides, solvents, inks and dyes, pigments, and herbicides.

25. The article of claim 1 wherein the detecting system comprises a binder, a plurality of particles and an indicator dye.

26. The article of claim 25 wherein the binder is a hydrophilic or hygroscopic polymer binder.

27. The article of claim 25 wherein the plurality of particles are a plurality of alumina particles having an average diameter in a range of from about 0.01 nanometer to about 1000 nanometers.

28. The article of claim 25 wherein the indicator dye is a polyazamacrocyclic transition metal complex.

29. The article of claim 28 wherein the transition metal is platinum or palladium.

30. The article of claim 25 wherein the detecting system comprises a patterned coating on at least one of the facestock film surfaces.

31. The article of claim 25 wherein the binder is present in the detecting system in an amount in a range of from 10 weight percent to 90 weight percent, the indicator dye is present in an amount in a range of from about 0.01 weight percent to about 5 weight percent, and the plurality of particles is present in an amount in a range of from about 10 weight percent to about 90 weight percent.

32. The article of claim 1 wherein the facestock film comprises paper or a polymeric film.

33. The article of claim 1 wherein the detecting system comprises a conductive ink layer comprising a conductive ink, electrical leads in electrical communication with the conductive ink layer, and an electrical signal monitoring device in electrical communication with the electrical leads, wherein the detecting system is responsive to contact with the analyte by indicating that such contact has occurred, the contact indication being a decrease in the amount of the conductive ink in the conducting layer, and the decrease in the amount of conducting ink in the conductive ink layer resulting in an increase in the electrical resistance of the conductive layer, and thereby generating a electrical signal to indicate that such contact has occurred.

34. An article for detecting an analyte, comprising:
a flexible facestock film having first and second substantially planar surfaces, wherein the first surface of the facestock film defines a micro-structure;
an adhesive layer having first and second substantially planar surfaces, wherein the adhesive layer first surface is adhered to the facestock film second surface; and
an immunoassay device is adhered to the facestock film first surface and oriented relative to the micro-structure, and the immunoassay device comprises:
a capture antibody layer comprising a species of capture antibodies, supported in a predetermined pattern on the facestock film first surface; and
a porous or perforate detector antibody layer, comprising a species of detector antibodies corresponding to the capture antibodies, the detector antibody layer overlying the capture antibody layer, whereby simultaneous binding of the capture antibodies and the corresponding detector antibodies with the analyte results in detectable signal.

35. The article of claim 34 wherein the detector antibodies comprise chromogenic ligands.

36. The article of claim 34 further comprising an absorbent layer in contact with the immunoassay device adhered to the facestock film first surface.

37. The article of claim 36 wherein the absorbent layer comprises a super-absorbent polymer.

38. An article for detecting an analyte, comprising:
a facestock film having first and second substantially planar surfaces;
an adhesive layer having first and second substantially planar surfaces, the adhesive layer first surface adhered to the facestock film second surface;
an immunoassay device supported on the facestock film first surface, the immunoassay device comprises:
a capture antibody layer comprising a species of capture antibodies, applied in a predetermined pattern on the facestock film first surface; and
a porous detector antibody layer, comprising a species of detector antibodies corresponding to the capture antibodies, the detector antibody layer overlying the capture antibody layer, whereby simultaneously binding the capture antibodies and the corresponding detector antibodies with the analyte results in the appearance of a visual signal; and
a hydrophilic or hygroscopic layer in contact with the immunoassay device.

39. The article of claim 38 wherein the fluid absorbing layer comprises an absorbent material selected from super-absorbent polymers, starch, and polyvinyl polymers, silicone-organic copolymer elastomers, activated alumina, calcium carbonate and silica gel.

40. A food package comprising the article of claim 38.

41. An article for detecting the presence of an analyte from a packaged material adjacent to a packaging substrate having an inner surface and an outer surface, comprising:
means for detecting the presence of an analyte, wherein the presence of the analyte is indicative of a condition of the associated packaged material; and
means for mounting the detecting means on the packaging substrate inner surface.

42. A detector system for detecting an analyte, comprising:
a transition metal complex;
a hydrophilic and water insoluble binder; and
a plurality of particles, wherein the transition metal complex, the binder and the particles form a layer that is responsive to contact with the analyte by indicating that such contact has occurred.

43. The system of claim 42 wherein the particles comprise alumina.

44. The system of claim 43 wherein the particles have an average diameter in a range of from about 0.01 nanometers to about 1000 nanometers, and the particle average diameter is selected to control the osmotic flow of fluid or vapor through the layer.

45. The system of claim 42 wherein the particles comprise titanium dioxide.

46. The system of claim 45 wherein the particles have an average diameter in a range of from about 0.01 nanometers to about 1000 nanometers, and the particle average diameter is selected to control the osmotic flow of fluid or vapor through the layer.

47. The system of claim 42 wherein the transition metal complex comprises a polyazamacrocyclic transition metal complex.

48. The system of claim 42 wherein the transition metal complex comprises palladium or platinum, and further comprises fluorescein.
